(19) United States
(12) Patent Application Publication
(10) Pub. No.: US 2022/0025309 A1
(43) Pub. Date: Jan. 27, 2022

(54) Microfluidic Device Facilitating Perfusion of Mammalian and Human Tissue Constructs

(71) Applicant: Cellink AB, Göteborg (SE)

(72) Inventors: Edvin Atic, Västra Frölunda (SE); Adel Itedal Namro Redwan, Skepplanda (SE); Hector Martinez, Göteborg (SE); Erik Gatenholm, Göteborg (SE)

(73) Assignee: Cellink AB, Göteborg (SE)

(21) Appl. No.: 17/311,472

(22) PCT Filed: Dec. 6, 2019
(86) PCT No.: PCT/EP2019/084086

§ 371 Date: Jun. 7, 2021

(51) Int. Cl. C12M 3/00; C12M 1/00; C12M 3/06; C12N 5/00; B33Y 10/00; B33Y 80/00

(52) U.S. Cl. CPC C12M 21/08; C12M 29/10; C12M 23/16; C12N 2513/00; C12N 5/0062; B33Y 10/00; B33Y 80/00; C12M 23/22

(57) ABSTRACT

A perfusion device for printing a 3D construct with internal channels is disclosed. The device comprises a housing with an internal chamber, the chamber configured to hold the construct, and having a bottom plate configured to support the construct, the chamber comprising at least one inlet or outlet configured to introduce a liquid from outside the chamber into the chamber or vice versa. The at least one inlet or outlet comprises an connector adapted to connect or align the one or more internal channels within the 3D construct to the inlet or outlet when the 3D construct is present in the chamber. Further a method for printing a 3D construct with at least one internal channel is disclosed.

### TECHNICAL FIELD

The present invention relates to the emerging fields of 3D bioprinting and functional tissue engineering. More specifically, embodiments of the invention relate to a perfusion device, as well as related methods for printing and use of a 3D construct with improved perfusion capability.

### BACKGROUND

In tissue engineering, the need for hierarchical assembly of three-dimensional (3D) tissues has become increasingly important, considering that new technology is essential for advanced tissue fabrication. 3D cell printing has emerged as a powerful technology to recapitulate the microenvironment of native tissue, allowing for the precise deposition of multiple cells onto the pre-defined position. To create a thick tissue that can accommodate a high density of cells, nutrient and gas exchange needs to be effective and reach every cell throughout the tissue. For this purpose, the tissue engineer needs to vascularize the tissue, however this has proven challenging.

Further, using common hydrogels, such as GelMA and the sacrificial bioink Pluronic F-127 has previously often led to great difficulties. Since hydrogels are hydrophilic and pluronics is hydrophobic, co-printing them is difficult and would usually result in failure.

There is thus a need for a method and a device for printing a 3D construct with improved perfusion capability.

### SUMMARY OF THE INVENTION

The above-mentioned problems are overcome by the present invention according to the independent claim. Preferred embodiments are set forth in the dependent claims.

To create a thick tissue or construct that can accommodate a high density of cells, nutrient and gas exchange needs to be effective and reach every cell throughout the tissue. For this purpose, it is advantageous for the tissue engineer to be able to vascularize the tissue. The approach disclosed herein includes the printing of one or several larger, primary vessels with a relatively simple geometry, from which a microvasculature is able to develop during incubation. Herein is disclosed a method and device for manufacturing a 3D bioprinted construct.

A perfusion device for printing a 3D construct with internal channels comprises
a housing with an internal chamber, the chamber configured to hold the construct, and comprising at least one inlet or outlet configured to introduce a liquid from outside the chamber into the chamber or vice versa,
the chamber having a bottom plate configured to support the construct comprising the one or more internal channels at least during printing of the 3D construct,
wherein each of the at least one inlet or outlet comprises a connector adapted to connect or align the one or more internal channels within the 3D construct to the inlet or outlet when the 3D construct is present in the chamber.

The perfusion device may further comprise a cover, e.g. made of glass, plexiglas or other suitable material, configured to enclose the chamber such that the chamber becomes essentially fluid-tight.

Further, the perfusion device may be provided with at least one perfusion passageway in connection with the inlet or outlet, adapted for fluid connection with the internal chamber. The passageways may be provided at the external end to luer or other suitable connectors, for connection with tubing connected with a suitable perfusion system.

A kit is disclosed, comprising the above described perfusion device, and further comprises a biomaterial adapted to be used for bioprinting, a sacrificial bioink, optionally at least one bioprinting nozzle adapted for bioprinting the biomaterial. Such a kit may further comprise suitable Luer connectors.

A method for printing a 3D construct with at least one internal channel comprises the steps of
providing a chamber with at least one inlet or outlet comprising a connector adapted to connect to said at least one internal channel, and said chamber further comprising a bottom plate,
optionally providing a first biomaterial layer on the bottom plate,
printing one or more channels on the bottom plate or on the first layer of biomaterial using a sacrificial ink, said one or more channels being printed such that they connect to or align with a connector,
providing a second biomaterial layer covering the one or more channels.

The method may further comprise, in the case of providing a first biomaterial layer on the bottom plate, a step of dehydration of the biomaterial in the first biomaterial layer before printing the one or more channels. In such a case, the second biomaterial may be adapted to rehydrate the first biomaterial layer. The first and/or second biomaterial may comprise a scaffold or matrix material, such as a hydrogel, and further preferably living biological cells, such as cells suitable for further incubation and growth within the biomaterial. The cells may be incorporated or added to the biomaterial before, during and/or after printing of a construct.

The method can preferably further comprise a step of evacuating the sacrificial ink using suction or pressure, using a suitable fluid or air. Preferably, the bottom plate is provided in an internal chamber of a perfusion device as described above, wherein the internal chamber is provided with inlets and/or outlets. The channels are then printed such that they align with at least one of the inlets or outlets. Thus, the one or more channels may be evacuated after printing by applying suction or positive pressure, to an inlet or outlet. Positive pressure or suction may be applied e.g. by applying pressure with a gas, such as air, or with a fluid or liquid, such as saline solution or other fluid.

By use of the above device and method, it is now possible to manufacture channeled tissues or constructs using biomaterials, preferably directly in the perfusion device.

The printed construct may be stored and optionally incubated directly in the perfusion device. For example, the user may place the device in an incubator of suitable type to allow cellular material to grow and mature, and/or store it in a suitable location. The perfusion device is also suitable for placing in a microscope to view the vascular structure and any cellular or bio-material.

The resulting bioprinted construct will have a hollow tube-like structure within the construct, wherein the hollow tubes allow perfusion of the construct. Such construct may be connected to a perfusion solution of suitable type, to allow for effective perfusion of the construct. This may be performed directly in the perfusion device where the construct has been printed. For instance, using luer connectors and suitable tubing, a user may easily connect the perfusion device to a perfusion solution, and thus connect the bioprinted construct with perfusion solution that supports the further incubation and development of a tissue with emerging microvasculature. For example, a nutrient solution may be used for perfusion, such that e.g. cells in the construct are exposed to nutrients, allowing them to grow effectively. In another aspect, a saline or other physiological solution may be used in perfusion, for rehydration, influx of specific substances and/or removal of substances in the construct.

The present disclosure thus presents a new method and device, which makes it possible to easily craft channeled tissues or constructs using bioinks and sacrificial inks. Using a perfusion device, such as the device described above and below, luer connectors, and connectors, the bioprinted construct can easily both be manufactured and therafter be interconnected with perfusion tubing in a novel manner. This supports the further incubation and development of a tissue with emerging microvasculature.

Parallel to these technological advances, the search for an appropriate bioink that can provide a suitable microenvironment supporting cellular activities has been in the spotlight. Bioinks are often comprised of a low viscosity or temperature sensitive biomaterial blended with a thickening agent to impart printability while also preserving cell viability and biological activity. Thus, a composition for use in a perfusion device, system or method as described above comprises various biomaterials in combination with thickeners and cells to fabricate bioprinted tissue models. Sacrificial inks allow the creation of hollow structures enabling perfusion for use in in vitro culture, tissue development, transplantation, and drug screening and development.

### FIGURES

FIG. 1A illustrates a vertical cross-sectional view of one aspect of a perfusion device as disclosed herein.

FIG. 1B illustrates the perfusion device of FIG. 1a with an exemplary bioprinted channel template.

FIG. 2A illustrates a horisontal cross-sectional view a perfusion device as disclosed in FIG. 1A.

FIG. 2B illustrates the perfusion device of FIG. 2A with an exemplary bioprinted channel template.

FIG. 3A illustrates a top view of a perfusion device as disclosed in FIG. 1A.

FIG. 3B illustrates a top view of the perfusion device as shown in FIG. 3A with an exemplary bioprinted channel template.

FIG. 4A illustrates a perspective view of another aspect of a perfusion device.

FIG. 4B illustrates a vertical cross-sectional view of the perfusions device of FIG. 4A.

FIG. 5 illustrates a horisontal cross-sectional view of the perfusions device of FIG. 4A.

### DESCRIPTION

Embodiments of the invention rely on the discovery that the use of a perfusion device, preferably with internal channel connectors, attached via tubing to an external perfusion system may be used for effective perfusion of bioprinted human tissues and scaffolds, as well as other tissues and 3D bioprinted constructs. Further, the combination of two polymers, one being a biomaterial (mammalian, plant based, or microbially derived) and a polysaccharide hydrogel-based thickener, and the other a sacrificial ink, such as pluronics with or without cells, may be used to create a 3D construct with hollow channels within the construct, which may be used for effective perfusion of the construct. Furthermore, the 3D construct with internal channels may be printed directly in the perfusion device itself, which saves both time and effort for the user. Furthermore, the 3D construct may advantageously be kept in the perfusion device while incubating and storing the construct, during perfusion as well as during any following investigations, such as viewing the printed material under a microscope.

Reference will now be made in detail to various exemplary aspects of the invention. It is to be understood that the following discussion of exemplary aspects is not intended as a limitation on the invention. Rather, the following discussion is provided to give the reader a more detailed understanding of certain aspects and features of the invention.

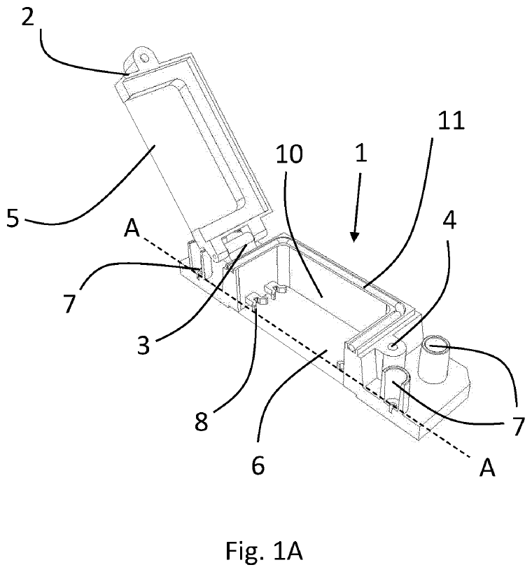

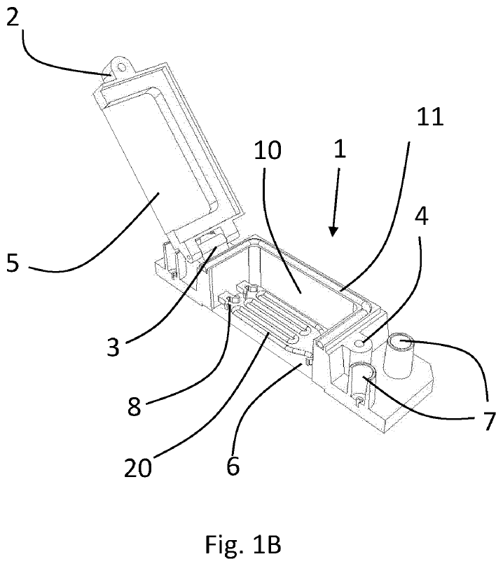

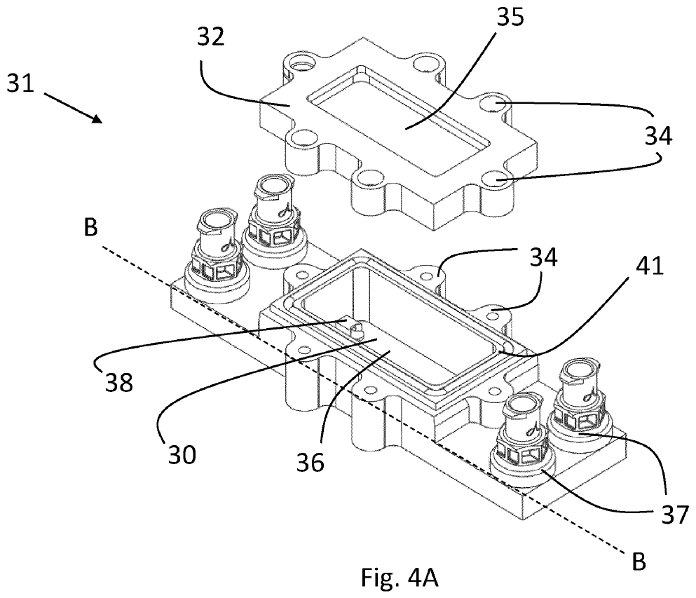

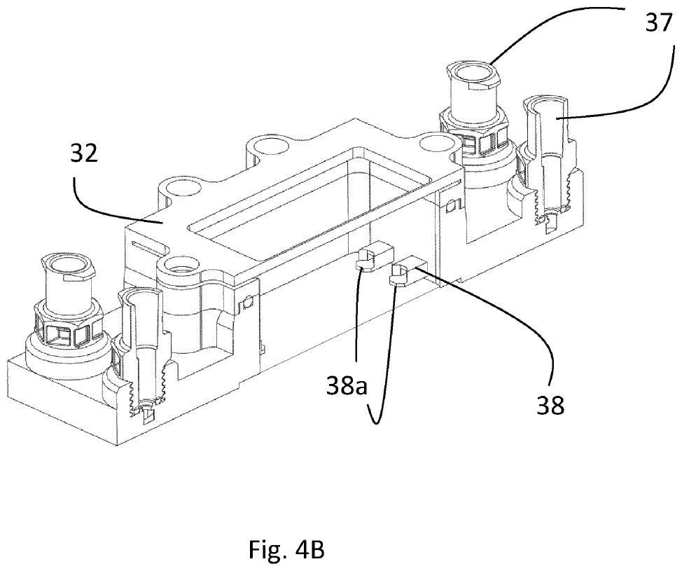

A perfusion device 1, 31 for printing a 3D construct with one or more internal channels is illustrated in FIG. 1A in perspective cross-sectional view. Another perfusion device is illustrated in perspective view in FIG. 4A, and in a perspective vertical cross-sectional view in FIG. 4B. These are two non-limiting examples of perfusion devices, and both comprise
a housing with an internal chamber 10, 30, the chamber configured to hold a 3D construct,
the chamber 10, 30 comprising at least one inlet or outlet configured to introduce a liquid from outside the chamber into the chamber or vice versa,
the chamber having a bottom plate 6, 36 configured to support the 3D construct comprising one or more internal channels at least during printing of the 3D construct,
wherein the at least one inlet or outlet comprises a connector 8, 38 adapted to connect or align the one or more internal channels within the 3D construct to the inlet or outlet when the 3D construct is present in the chamber.

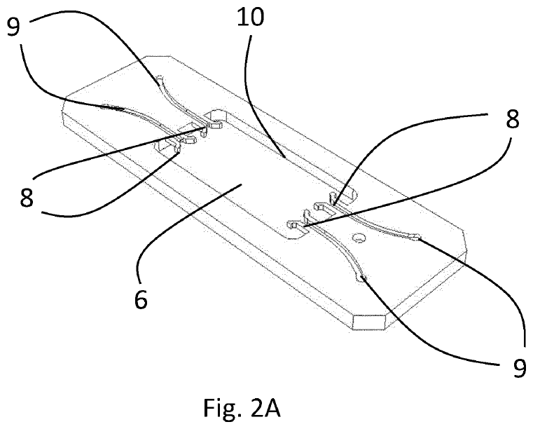

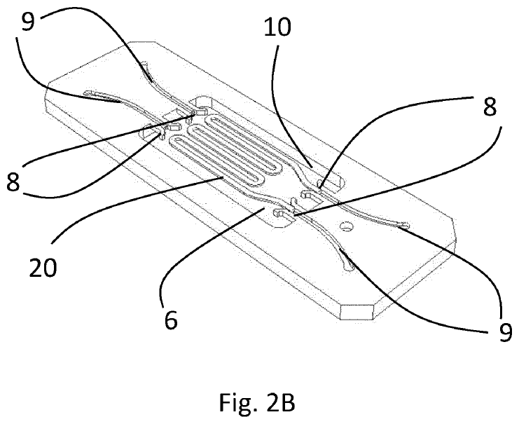

Further, the perfusion device may be provided with at least one perfusion passageway 9, 39 in connection with the inlet or outlet, adapted for fluid connection with the internal chamber 10, 30. This is illustrated in in one example in FIG. 2A which shows a horizontal crossectional view of the perfusion device of FIG. 1A along lines A-A. Notably, the view in FIG. 2A is of another crosssectional view than that seen in FIG. 1A, i.e. a cross-sectional view being perpedicular to that seen in FIG. 1A. Thus is seen, in FIG. 2A, the bottom part of chamber 10, the bottom plate 6, as well as the perfusion passageways 9. It may be understood from the combination of FIGS. 1A and 2A that the perfusion passageways 9 lead from the chamber 10 to external connectors 7.

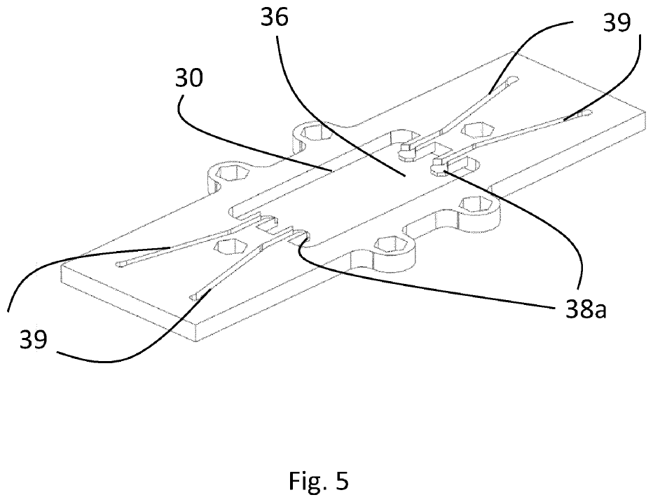

Similarly the perfusion device of FIG. 4A may be seen in a horizontal crossectional view along lines B-B in FIG. 5. The perfusion passageways 39 lead from the chamber 30 to external connectors 37. The bottom plate 30 forms the bottom of the chamber 30.

The passageways may be connected at the external end to suitable connectors 7, 37 for connection with tubing connected with a suitable perfusion system. Such external connectors may comprise e.g. Luer connectors, or screw-nuts or wing-nuts, the latter being advantageous when attaching elastic tubing to the external connectors of the perfusion device.

The perfusion device 1, 31 is thus adapted for directly printing a 3D construct with one or more internal channels, and for a following incubation and/or perfusion of the printed construct. In other words, the device may be placed on the printbed or print area of a bioprinter, and a construct manufactured directly in the chamber. The bottom plate of the chamber is thus configured to support the 3D construct comprising the one or more internal channels at least during printing of the 3D construct.

A printed 3D construct may be kept within the chamber after printing and the entire perfusion device moved, if needed, to e.g. a lab bench or a cell incubator, for perfusion of the construct via the printed channels, and/or any incubation necessary for growth of biological material. The device is also suitable for storage of the printed construct for later use.

The perfusion device may for instance be used to model vascular structures to investigate advanced cellular and tissue phenomena.

The device may comprise any suitable number of inlets/outlets, preferably at least two, or more preferable at least three inlets and outlets. In some aspects, the perfusion device comprises two inlets and two outlets, or three inlets and three outlets. However, any combination is feasible. Using multiple inlets and outlets allows for more effective perfusion of the final construct, as well as adding possible endpoints of printed channels, thus allowing a large number of possible channel configurations in the construct.

As mentioned above, the at least one inlet or outlet comprises an connector 8, 38 adapted to connect or align the one or more internal channels within a 3D construct to the inlet or outlet when the 3D construct is present in the chamber.

In a first aspect, the perfusion device may comprise an anchoring connector 8 (FIG. 1A and 2A) which becomes embedded in the 3D construct inside the chamber during printing, and ensures that the construct is held securely both during printing and during following perfusion of the construct. In the figures the anchoring connector is illustrated as having two splayed extensions or hooks. However any suitable configuration is conceivable that act as an anchoring structure, such as different types of extending members. The anchoring connectors 8 prevent slipping or loosening of the construct from the chamber. In addition, this also ensures that the formed channels are held in alignment with the perfusion passageway in connection with the inlet or outlet.

In another aspect, as shown in FIG. 4B and FIG. 5, a connector 38 may comprise a distancing element 38a adapted to assist or provide guidance to a 3D printer in placing a material for the internal channels at suitable height from the bottom plate 36, such that the internal channels will align with the inlet or outlet. Thus, such distancing elements, here illustrated as a small platform 38a at each inlet or outlet, assures that the channels are printed at essentially the same height, and at a certain distance from the bottom plate. In the illustrated example, the platform 38a makes it easy to position the printing nozzle correctly prior to printing sacrificial ink of a the channel.

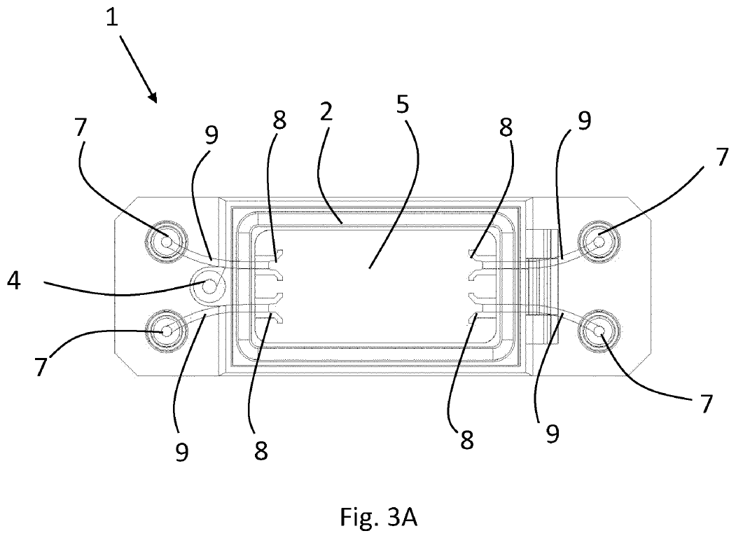

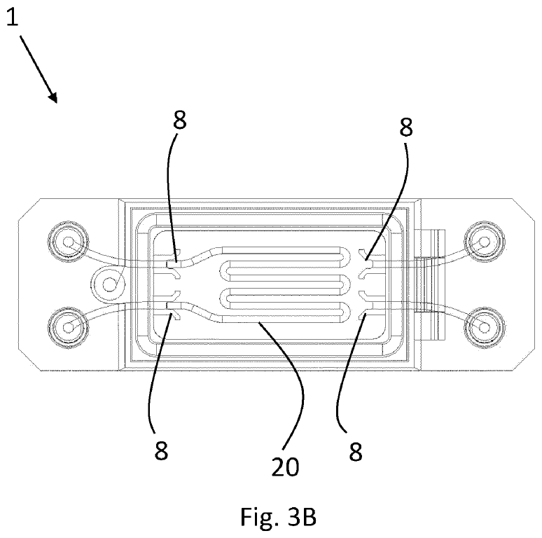

The chamber 10, 30 may comprise a cover 2, 32. Furthermore, a sealing means 11, 41 may be provided either on the cover and/or on the rim of the chamber. Such sealing means may be an 0-ring or the like around the edge such that when the cover is closed, the chamber becomes essentially fluid-tight. Such sealing means may made of a suitable material, such as rubber, silicone plastic, Nitril, Viton, Kalrez, fluorocarbon, or polyacrylate. This would for instance inhibit humidity and/or liquid to escape from the chamber, with exception of via an inlet or outlet. FIG. 3A illustrates a top view of the perfusion device of FIG. 1A, with the cover 2 closed.

As is understood from the above, the chamber 10, 30 must be open during bioprinting of a construct. A cover 2, 32 may be attached by any known means, such as a hinge 3, one or several screws and corresponding screwholes 4, 34 or by snaplock, or any combination thereof. The cover 32 may be completely removable, such as is shown in FIG. 4A, which frees up space and allows more room for any moving parts of a bioprinter, and removes the risk of collision of any bioprinter parts with the cover during printing.

The cover is preferably at least partly transparent, e.g. by providing e.g. a glass. plexiglass or plastic window 5, 35 in the cover. More preferably, the cover is essentially fully transparent over most of its surface. This allows a user to see into the chamber and view the construct including the channels, even when the chamber is closed.

Furthermore, the bottom plate 6, 36 of the chamber is preferably at least partly transparent, more preferably essentially fully transparent. Similar materials as for the window in the cover may be used, e.g. glass, plexiglass or plastic. This also allows the user to see into the chamber from the bottom. If both the cover and bottomplate are at least partly transparent, a good view of the channels is provided from either direction. Furthermore, the device may be placed on or under a lamp to provide a better view. The construct within the chamber may preferably be viewed in a microscope. The perfusion device may thus be used as a bioreactor for channel perfusion and visual inspection of fully or partially bioprinted constructs or tissues.

The perfusion device as described herein is preferably made in a suitable biocompatible material. Examples include polymer resin, glass, stainless steel, polystyrene etc.

The perfusion device is preferably sterilizable, for instance by autoclaving, heating, ethanol treatment or UV light.

The size of the perfusion device and of the chamber itself may be any suitable size usable in bioprinting or perfusion. For example a perfusion device may be 5-15 cm long and 2-5 cm wide, with a chamber being 3-12 cm long, 2-4 cm wide and 1-4 cm tall. However, both smaller and larger sizes are conceivable as well as other overall shapes.

A kit is disclosed, comprising the above described perfusion device, and further comprises a biomaterial adapted to be used for bioprinting, a sacrificial bioink, optionally at least one bioprinting nozzle adapted for bioprinting the biomaterial. Such a kit may further comprise suitable Luer connectors. Suitable biomaterials and sacrificial bioinks will be described further below.

A method for printing a 3D construct with at least one internal channel comprises the steps of
providing a chamber with at least one inlet or outlet comprising a connector adapted to connect to or align with said at least one internal channel, and said chamber further comprising a bottom plate,
optionally providing a first biomaterial layer on the bottom plate,
printing one or more channels on the bottom plate or on the first layer of biomaterial using a sacrificial ink, said one or more channels being printed such that they connect to or align with a connector,
providing a second biomaterial layer covering the one or more channels.

In one aspect, a first biomaterial layer is printed on the bottom plate. The channels are then printed on the first biomaterial layer. In such a case, the method may further comprise, a step of dehydration of the biomaterial in the first biomaterial layer before printing the one or more channels. In a further aspect, the second biomaterial may be adapted to rehydrate the first biomaterial layer.

In another aspect, the one or more channels are printed directly on the bottom plate, such that that they connect to a connector. Therafter a layer of biomaterial is provided to cover the one or more channels.

In any case, the biomaterial provided to cover the the one or more channels may be provided over essentially only the channels, or may be provided over essentially the entire surface of the previously provided provided material.

The first and/or second biomaterial may comprise a scaffold or matrix material, such as a hydrogel, and further preferably living biological cells, such as cells suitable for further incubation and growth within the biomaterial. The cells may be incorporated or added to the biomaterial layer(s) before, during and/or after printing of a construct.

In one aspect, the biomaterial may comprise GelMA (Gelatin Methacryloyl) or other compatible material. In another aspect, the sacrificial ink may comprise Pluronic (Pluronic® F-127, powder, BioReagent, suitable for cell culture, provided by Sigma-Aldrich) or other compatible material.

The method can preferably further comprise a step of evacuating the sacrificial ink using suction or pressure, using a suitable fluid or air. Preferably, the bottom plate is provided in an internal chamber of a perfusion device as described above, wherein the internal chamber is provided with inlets and/or outlets. The channels are then printed such that they align with at least one of the inlets or outlets. Thus, the one or more channels may be evacuated after printing by applying suction or positive pressure, to an inlet or outlet. Positive pressure or suction may be applied e.g. by applying pressure with a gas, such as air, or with a fluid or liquid, such as saline solution or other fluid.

In one aspect, the method is performed using at least one of the following conditions:
the temperature during the 3D bioprinting is in the interval from 4° C. to 40° C. and most appropriately at approximately 37° C.; or
the printing pressure during the 3D bioprinting is in the interval from 1 to 200 kPa, and preferably below 50 kPa, and even more preferably in the interval from 5-25 kPa, when bioprinting with cells.

One example of use of the method is described below.

A sterile perfusion device, such as that shown in the Figures, is provided with a thin layer of biomaterial, preferably GelMA, to cover the glass bottom. The GelMA is dehydrated by air drying from 1 h to 24 h, or may also include heat assisted air drying. Therafter a channel- or tube-shaped construct 30 is printed on the dried layer of GelMA, using a sacrificial ink, such as Pluronics. One example is illustrated in FIG. 1B, 2B, and 3B. A top layer of GelMA in provided to; a) cover the Pluronics tube and b) rehydrate the GelMA in the bottom layer. This step can be performed with or without cells. Thereafter, the GelMA is crosslinked using light induced photo crosslinking or enzymatic assisted crosslinking.

Media and cells may also be added on top of the GelMA. The chamber may be sealed using the lid if required.

The device is then preferably cooled to around 4 degrees Celsius, to liquify the sacrificial ink, i.e. Pluronics. Then the sacrificial can easily be evacuated by suction to create a channel. This may be done with a syringe or by connecting suction to the inlet/outlet channels. The channel can be equipped with syringe-assisted or pump-assisted perfusion (e.g. peristaltic or diaphragm pump).

The materials may or may not be seeded with cells.

The device may then be incubated according to the required experimental conditions.

Suitable bioink compositions comprise biocompatible botanical polysaccharide, such as acacia gum, tara gum, glucomannan, pectin, locust bean gum, guar gum, carrageenan, and tragacanth, and/or a microbial or fungal-produced polysaccharide hydrogel-based thickener known as biogums (such as xanthan gum, gellan gum, diutan gum, welan gum, and pullalun gum), with or without cells, together with a mammalian, plant, microbial-derived, or synthetic hydrogel for bioprinting of human tissue analogues and scaffolds under physiological conditions. Furthermore, the bioink composition can be supplemented through the addition of auxiliary proteins and other molecules such as extracellular matrix components, Laminins, growth factors including super affinity growth factors and morphogens. In addition, a sacrificial ink including Pluronics enables the formation of hollow tubes, interconnected with perfusion tubing in a novel manner, that supports the further incubation and development of a tissue with emerging microvasculature with nutrient and gas exchange.

The physiological conditions are related to 3D bioprinting parameters which are cytocompatible (e.g. temperature, printing pressure, nozzle size, bioink gelation process). According to one example, the combination of a botanical gum polysaccharide hydrogel together with mammalian, plant, microbial or synthetically derived hydrogels exhibited improvement in printability, cell function and viability compared to tissues printed with bioink not containing these botanical gums and/or a microbial or fungal-produced polysaccharide. Aspects thus include products (e.g. human tissue specific bioinks) and methods (e.g. physiological printing conditions), as well as several applications.

One aspect relates to a device and a bioink composition for use in 3D bioprinting comprising:
(i) a plant-produced thickener component (botanical gum),
(ii) a microbial or fungal-produced thickener component
(iii) a mammalian, plant, microbial, or synthetically derived biomaterial component,
(iv) a sacrificial ink, and
(v) an auxiliary component
   wherein the composition is provided with or without cells.

In some aspects the composition is provided with cells, preferably human cells.

In some aspects, the acacia-gum is produced from plant species, including one or more of:
1. Acacia nilotica
2. Acacia Senegal
3. Vachellia (Acacia) seyal
4. Combretum, Albizia

In some aspects, the tara gum is produced from T. spinos.

In some aspects, the glucomannan is produced from Amorphophallus konjac.

In some aspects, the pectin is produced from rinds of lemons, oranges, apples.

In some aspects, the locust bean gum is produced from Ceratonia siliqua.

In some aspects, the guar gum is produced from Cyamopsis tetragonolob.

In some aspects, the carrageenan is produced from the Chondrus crispus (Irish moss).

In some aspects, the tragacanth is produced from legumes of the genus Astragalus including one or more of:
(i) A. adscendens
(ii) A. gummifer
(iii) A. brachycalyx,

In some aspects the ratio of botanical gums versus biomaterial by weight (w:w) is in the interval from 5:95 to 95:5, and preferably the ratio of botanical gums versus biomaterial is in the interval from 80:20 to 20:80 w:w.

In some aspects, the botanical gums thickener component has a concentration in the interval from 0.5 to 50% weight by volume (w/v). This concentration level is relevant both as initial and final concentration, and after dilution with other components of the composition.

In some aspects the composition is provided with cells, preferably human cells.

In some aspects, the xanthan gum is produced from Gram negative bacteria of the Xanthomonads genus, including one or more of:
(i) X. campestris
(ii) X. fragaria 1822
(iii) X. arboricola
(iv) X. axonopodis
(v) X. citri
(vi) X. fragaria
(vii) X. gummisudans 2182
(viii) X. juglandis 411
(ix) X. phaseoli 1128
(x) X. vasculorium 702

In some aspects, the gellan gum is produced from Gram negative bacteria Sphingomonas Eldoda of the Sphingomonas genus. In some aspects, the Curdlan gum is produced from Gram negative bacteria of the Alcaligenes faecalis of the Alcaligenes genus. In some aspects, the Welan gum is produced from Gram negative bacteria of the Alcaligenes genus. In some aspects, the Pullulan gum is produced from the fungus Aureobasidium pullulans. In some aspects the ratio of xanthan gum versus biomaterial by weight (w:w) is in the interval from 5:95 to 95:5, and preferably the ratio of xanthan gum versus biomaterial is in the interval from 80:20 to 20:80 w:w. In some aspects, the xanthan gum thickener component has a concentration in the interval from 0.5 to 10% weight by volume (w/v). This concentration level is relevant both as initial and final concentration, and after dilution with other components of the composition.

In some aspects, the mammalian, plant, microbial or synthetically derived biomaterial is chosen from at least one of the following constituents for cross-linking purposes and/or to contribute to rheological properties of the bioink, such as hydrocolloids or thickening and gelling agents: collagen type I, collagen and its derivatives, gelatin methacryloyl, gelatin and its derivatives, fibrinogen, thrombin, elastin, alginates, agarose and its derivatives, glycosaminoglycans such as hyaluronic acid and its derivatives, chitosan, low and high methoxy pectin, gellan gum, diutan gum, glucomannan gum, carrageenans, nanofibrillated cellulose, microfibrillated cellulose, crystalline nanocellulose, carboxymethyl cellulose, methyl and hydroxypropylmethyl cellulose, bacterial nanocellulose, and/or any combination of these constituents.

The composition according to the present disclosure, wherein the bioink comprises additional biopolymers for cross-linking purposes and/or to contribute to rheological properties of the bioink, such as hydrocolloids or thickening and gelling agents.

The composition according to the present disclosure, wherein the additional biopolymer, hydrocolloid or thickening and gelling agent is chosen from alginates, hyaluronic acid and its derivatives, agarose and its derivatives, chitosan, fibrin, gellan gum, silk nanofibrillated cellulose, microfibrillated cellulose, crystalline nanocellulose, bacterial nanocellulose, carrageenans, elastin, collagen and its derivatives as well as gelatin and its derivatives.

In some aspects the concentration of mammalian, plant, microbial or synthetically derived biomaterials is in the interval from 0.5 to 50% (w/v), preferably from 0.5 to 10% (w/v) , and the concentration of cells is in the interval from 0.1 million/ml to 150 million/ml.

In some aspects, the mammalian, plant, microbial or synthetically derived biomaterials include one or more of:
a. Alginate and its derivatives
b. Agarose and its derivatives
c. Gelatin and its derivatives
d. Collagen and its derivatives
e. Fibrin and its derivatives
f. Hyaluronic acid
g. Basement membrane matrix
h. Laminins
i. Fibronectin and its derivatives
j. Heparan sulfate proteoglycans
k. Cellulose and its derivatives
l. Pectin and its derivatives
m. Chitosan and its derivatives
n. Silk and its derivatives
o. Polyethylene glycol and its derivatives
p. Poly (vinyl alcohol)-based hydrogels
q. Poly(N-isopropylacrylamide) (PNIPAM)
r. Poly(2-hydroxypropyl methacrylate (PHPMA)
s. Poly(2-hydroxyethyl methacrylate) (PHEMA)

In some aspects, the composition is provided under physiological conditions.

In some aspects, the composition is provided so that at least one of the following conditions are met:
a. a pH-value for the composition in the interval from 5-8, including 5-7, 6-8, 7-8, and preferably about 7;
b. the osmolarity of the composition is in the interval from 275 to 300 mOsm/kg, including 275-295, 280-295, 280-300, 285-300, preferably about 295 mOsm/kg.

In some aspects, the auxiliary components may be in concentrations ranging from 0.5% to 50% and may include one or more of:
a. Fibronectin and its derivatives
b. Collagen and its derivatives
c. Extracellular matrix
d. Basement membrane matrix
e. Fibrin and its derivatives
f. Elastin and its derivatives
g. Glycosaminoglycans and its derivatives including
   a. Hyaluronic acid
   b. Chondroitin sulfate
   c. Dermatin sulfate
   d. Heparin sulfate
   e. Keratin sulfate
h. Laminin and its derivatives
i. Small Molecules
j. Peptides
   a. Adhesive
   b. Differentiation
   c. Morphogenic
k. Lysozyme
l. Growth factors and Morphogens including
   a. Proliferative
   b. Differentiation
      i. Chondrogenic
      ii. Fibrogenic
      iii. Myogenic
      iv. Cardiomyogenic
      v. Neurogenic
      vi. Heptagenic
      vii. Pancreatic
      viii. Renal
      ix. Intestine
      x. Dermal
      xi. Osteogenic
      xii. Oncogenic
   c. Stemness Maintenance
      i. Chondrogenic
      ii. Fibrogenic
      iii. Myogenic
      iv. Cardiomyogenic
      v. Neurogenic
      vi. Heptagenic
      vii. Pancreatic
      viii. Renal
      ix. Intestine
      x. Dermal
      xi. Osteogenic
      xii. Oncogenic
m. Fluorescently labeled proteins and biomolecules

A second aspect relates to a method for employing a microfluidic device and 3D bioprinting of human tissue comprising bioprinting the composition as described herein, thereby combining botanical gum and/or a microbial or fungal -based bioink, and a biomaterial derived from mammalian, plant, microbial or synthetic sources, with human or mammalian cells and a sacrificial ink such as Pluronics to allow for perfusion.

A third aspect relates to a method for 3D bioprinting of at least one scaffold comprising bioprinting the composition as disclosed herein, thereby combining a botanical gum and/or a microbial or fungal -based thickener and a mammalian, plant, microbial or synthetic derived biomaterial. One aspect relates to the cultivation of the bioprinted constructs in a microfluidic device, with a connector attached using tubing to an external perfusion system. In some aspects, the method(s) for bioprinting as disclosed herein is/are performed under physiological conditions.

In some aspects related to the methods for bioprinting as disclosed herein at least one of the following conditions are met during 3D bioprinting:
a. the temperature during the 3D bioprinting is in the interval from 4° C. to 40° C., including 10° C. to 40° C., 20° C. to 40° C., and 30° C. to 40° C. and most appropriately at 37° C.; or
b. the printing pressure during the 3D bioprinting is in the interval from 1 to 200 kPa, and preferably below 50 kPa, including 5-45 kPa, 10-35 kPA, and 5-40 kPa, and even more preferably in the interval from 5-25 kPa, when bioprinting with cells

A further aspect relates to a bioprinted tissue or organ prepared by the method for 3D bioprinting with human cells as disclosed herein.

Yet another aspect relates to the bioprinted construct, tissue or organ as disclosed herein, for use in therapeutic applications of including treatment of liver diseases, metabolic diseases, diabetes, heart diseases, kidney diseases, skin defects, bone defects, bone and soft tissue sarcomas, lung diseases, vessels repair, intestinal diseases, retinal defects, bladder diseases, prostate diseases, tissue fibrosis (e.g. liver, kidney, intestine, lung, skin), cancer in any tissue, such as hepatocellular carcinoma, metastases in any tissue, such as the liver, colon or pancreas, colon cancer, lung cancer, liver cancer, pancreatic cancer, and cancer in any other tissue, wherein a bioprinted construct, tissue or organ according to the above is perfused with a fluid through at least one internal channel.

Another aspect relates to a method for treating liver diseases, metabolic diseases, diabetes, heart diseases, kidney diseases, skin defects, bone defects, bone and soft tissue sarcomas, lung diseases, vessels repair, intestinal diseases, retinal defects, bladder diseases, prostate diseases, tissue fibrosis (e.g. liver, kidney, intestine, lung, skin), cancer in any tissue, such as hepatocellular carcinoma, metastases in any tissue, such as the liver, colon or pancreas, colon cancer, lung cancer, liver cancer, pancreatic cancer, and cancer in any other tissue comprising wherein a bioprinted construct, tissue or organ according to the above is perfused with a fluid through at least one internal channel.

Still another aspect relates to a method for culturing the bioprinted tissue or organ as disclosed herein, wherein the bioprinted tissue or organ is cultured under physiological or pathological conditions.

In some aspects, at least two types of cells are co-cultured at different ratios. Ratios for cells in co-culture are chosen from: 1:1; 1:5, 1:10, 1:25, 1:50; 1:100, 1:150 and any range in between. In case of more than two cell types in culture the ratio is chosen from: 1:1:1; 1:1:5; 1:1:10; 1:1:50; 1:1:100 and any range in between.

In another aspect, the method of culturing is for the purpose of in vitro culture, disease modelling, drug screening, biomarker discovery, tissue models for drug development, substance testing and bioactive compound efficacy testing.

A further aspect relates to an in vitro culture prepared by the method for culturing as disclosed herein.

Another aspect relates to the use of the in vitro culture as disclosed herein for tissue development, disease development, drug screening and development and biomarkers. Yet another aspect relates to a bioprinted scaffold prepared by the method for 3D bioprinting as disclosed herein. Still another aspect relates to the use of the bioprinted scaffold as disclosed herein for wound healing.

A further aspect relates to a method for preparing recellularised tissue, comprising repopulating the bioprinted scaffold or construct as disclosed herein. Another aspect relates to a recellularised bioprinted tissue, produced by repopulating the bioprinted scaffold as disclosed herein with human cells.

Yet another aspect relates to a bioprinted tissue, scaffold or recellularised bioprinted tissue, further comprising growth factors.

Still another aspect relates to a method for promoting tissue repair, comprising implanting the bioprinted tissue, scaffold or recellularised tissue comprising growth factors as disclosed herein in a diseased tissue or organ.

Another aspect relates to a method of transplanting a bioprinted tissue, organ or scaffold as disclosed herein, wherein the bioprinted scaffolds and/or tissues are implanted into the diseased tissue or organ, such as ectopically implanted subcutaneously or intra-omentum or directly as tissue-patches into the diseased tissue or organ.

Still another aspect relates to a method of repairing a tissue or an organ, wherein the bioprinted scaffolds and/or tissues as disclosed herein are implanted as tissue-patches for improving wound healing.

Another aspect relates to a method of treating a disease in a tissue or an organ, wherein a bioprinted tissue as disclosed herein or a recellularised bioprinted tissue as disclosed herein is applied to the tissue or organ, such as by injection, implantation, encapsulation or extracorporeal application.

A further aspect relates to a method for disease modelling, comprising the steps of:
a. providing a bioprinted scaffold or construct as described herein;
b. mechanistic investigations;
c. determining the effect of a compound, drug, biological agent, device or therapeutic intervention on the scaffold or tissue.

Still another aspect relates to a bioprinted tissue, scaffold or recellularised bioprinted tissue for use in one or more of:
a. implantation in a diseased tissue or organ;
b. transplantation into a human or animal body, whereby the bioprinted scaffolds and/or tissues are ectopically implanted subcutaneously or intra-omentum or directly as tissue-patches into the diseased tissue or organ;
c. repairing a tissue or an organ, whereby the bioprinted scaffolds and/or tissues are implanted as tissue-patches for improving wound healing; and/or
d. treating a disease in a tissue or an organ, wherein a bioprinted tissue of claim or a recellularised bioprinted tissue is applied to the tissue or organ, such as by injection, implantation, encapsulation or extracorporeal application.

### Definitions

### Definition of Essential Terms and Claim Features.

“Microfluidic device” or “perfusion device”: A device for constructing and/or perfusing a material with a liquid comprising a container including a container, an internal chamber configured to hold the material with anchor connector, luer locks for the attachment of tubing to an external perfusion system.

“Biogum” refers to polysaccharides produced by bacteria or other microbials; examples of biogums include xanthan gum, gellan gum, diutan gum, welan gum, and pullalun gum. “Xanthan Gum” refers to a heteropolysaccharide with a primary structure that consists of pentasaccharide units consisting of two mannose, one glucuronic acid, and 2 glucose units. Xanthan consists of a backbone of glucose units with trisaccharide sidechains consisting of

Mannose-Glucuronic Acid-Mannose linked to everyone other glucose unit at the 0-3 position. “Gellan Gum” refers to a heteropolysaccharide with a primary structure that consists of tetrasaccharide units that consist of two glucose, one glucuronic acid, and one rhamnose unit. The backbone structure is glucose-glucuronic acid-glucose-rhamnose. “Diutan Gum” refers to a polysaccharide consisting of a repeating unit that is composed of six sugars. The backbone is made up of d-glucose, d-glucuronic acid, d-glucose, and l-rhamnose, and the side chain of two l-rhamnose. “Welan Gum” refers consists of repeating tetrasaccharide units with single branches of L-mannose or L-rhamnose. “Pullalun Gum” refers to a neutral polymer composed of α-(1,6)-linked maltotriose residues, which in turn are composed of three glucose molecules connected to each other by an α-(1,4) glycosidic bond.

“Mammalian, plant, microbial, or synthetic hydrogels” refers to any biocompatible polymer network that exhibits characteristics of a hydrogel. A hydrogel is a polymer network that has hydrophilic properties. Mammalian hydrogels consist of proteins or polymers derived from the various tissues, organs, and cells found in mammals including humans, porcine, bovine. Plant hydrogels consist of proteins or polymers derived from various plants including trees, algae, kelp, seaweed. Microbial hydrogels include polysaccharides produced by bacteria such as xanthan gum, gellan gum, diutan gum, welan gum, and pullalun gum. Synthetic hydrogels include polymers derived from polyethylene, polyethylene, polycaprolactone, polylactic, polyglycolic acid, and their derivatives.

In the context of the present disclosure, the term “bioprinted scaffold” refers to a bioprinted construct, structure or tissue printed with a composition without cells. On the other hand, the term “bioprinted tissue” refers to a bioprinted structure or tissue printed with a composition with cells.

“Botanical gum” refers to polysaccharides isolated from plants; examples of botanical gums include acacia gum, tara gum, glucomannan, pectin, locust bean gum, guar gum, carrageenan, and tragacanth. “Acacia Gum” refers to a heteropolysaccharide obtained from the Senegalia (Acacia) senegal and Vachellia (Acacia) seyal trees. This gum contains arabinogalactan which consists of arabinose and galactose monosaccharides that are attached to proteins creating what is known as arabinogalactan proteins. “Tara Gum” refers to a heteropolysaccharide isolated from T spinos of the Tara family consisting of a linear main chain of (1-4)-β-D-mannopyranose units attached by (1-6) linkages with α-D-galactopyranose units. “Glucomannan” refers to a straight-chain polymer, with a small amount of branching isolated from the roots of the konjac plant. The component sugars are β-(1→4)-linked D-mannose and D-glucose in a ratio of 1.6:1. “Pectin” refers to a heteropolysaccharide found in the primary cell walls of terrestrial plants. These include homogalacturonans are linear chains of α-(1-4)-linked D-galacturonic acid, rhamnogalacturonan II (RG-II), which is complex and highly branched polysaccharide, amidated pectin, high-ester pectin, low-ester pectin. “Locust bean gum” refers to high-molecular-weight hydrocolloidal polysaccharides, composed of galactose and mannose units combined through glycosidic linkages, which may be described chemically as galactomannan. It is dispersible in either hot or cold water, forming a sol having a pH between 5.4 and 7.0, which may be converted to a gel by the addition of small amounts of sodium borate. Locust bean gum is composed of a straight backbone chain of D-mannopyranose units with a side-branching unit of D-galactopyranose having an average of one D-galactopyranose unit branch on every fourth D-mannopyranose unit. “Guar gum” refers to an exo-polysaccharide composed of the sugars galactose and mannose. The backbone is a linear chain of β 1,4-linked mannose residues to which galactose residues are 1,6-linked at every second mannose, forming short side-branches.

“Carrageenan” refers to a polysaccharide isolated from red algae that are high-molecular-weight polysaccharides made up of repeating galactose units and 3,6 anhydrogalactose (3,6-AG), both sulfated and nonsulfated. The units are joined by alternating α-1,3 and β-1,4 glycosidic linkages. Three classes of Carrageenan are Kappa, Iota, and Lambda. Kappa forms stiff gels in the presence of potassium and is isolated from Kappaphycus alvarezii. Iota forms soft gels in the presence of calcium ions and is isolated from Eucheuma denticulatum. Lambda does not gel, and is used as a pure thickener. “Tragacanth” refers to a dried sap of several species of Middle Eastern legumes of the genus Astragalus, including A. adscendens, A. gummifer, A. brachycalyx.

“Mammalian, plant, microbial, or synthetic hydrogels” refers to any biocompatible polymer network that exhibits characteristics of a hydrogel. A hydrogel is a polymer network that has hydrophilic properties. Mammalian hydrogels consist of proteins or polymers derived from the various tissues, organs, and cells found in mammals including humans, porcine, bovine. Plant hydrogels consist of proteins or polymers derived from various plants including trees, algae, kelp, seaweed. Microbial hydrogels include polysaccharides produced by bacteria such as xanthan gum, gellan gum, diutan gum, welan gum, and pullalun gum. Synthetic hydrogels include polymers derived from polyethylene, polyethylene, polycaprolactone, polylactic, polyglycolic acid, and their derivatives.

“Bioprinting” refers to the utilization of 3D printing and 3D printing-like techniques to combine cells, growth factors, and biomaterials to fabricate biomedical parts that maximally imitate natural tissue characteristics. Generally, 3D bioprinting utilizes the layer-by-layer method to deposit materials known as bioinks to create tissue-like structures that are later used in medical and tissue engineering fields.

As used herein, “physiological conditions” include conditions (such as pH, osmolarity, temperature and printing/extrusion pressure) that are typical to the normal living environment for a culture or cells, such as, for human cells, a temperature around 37° C., such as in the interval from 35-39° C., a printing pressure in the interval from 1 kPa to 200 kPa, preferably below 25 kPa, a pH in the interval from 5-8, preferably about 7, and an osmolarity in the interval from 275 to 300 mOsm/kg, preferably about 295 mOsm/kg.

As used herein, “pathological conditions” include exposure of a culture or cells to inflammatory and/or carcinogenic conditions, e.g. recapitulating the disease.

As used herein, “co-culturing” cells means that cells of at least two types are cultured together.

In the context of the present disclosure, the term “bioprinted scaffold” refers to a bioprinted construct, structure or tissue printed with a composition without cells. On the other hand, the term “bioprinted tissue” refers to a bioprinted construct, structure or tissue printed with a composition with cells.

### Composition

A first aspect relates to the use of a microfluidic device with novel anchoring connectors, attached via tubing to an external perfusion system.

Herein is disclosed a bioink composition comprising a botanical gum and/or a microbial or fungal-produced -based thickener, and a mammalian, plant, microbial or synthetic derived biomaterial with or without cells depending on the application, with or without auxiliary components.

Method for bioprinting

Another aspect relates to a method for preparing bioprinted tissues or scaffolds that are suitable for use in the various products, uses and methods as disclosed herein.

In general, using a microfluidic device, the method for 3D bioprinting of human tissue (with cells) or scaffolds (without cells) comprises combining botanical gum thickener and/or a microbial or fungal-produced-thickener-based bioink, (with or without human cells), and human tissue-specific extracellular matrix (ECM) material, in combination with a sacrificial ink enabling evacuation followed by perfusion, wherein the 3D bioprinting is performed under physiological conditions.

The 3D bioprinted tissue or scaffold can be in the form of a grid, drop, tissue-specific shapes like hepatic lobule for liver etc., or the like. The 3D bioprinted tissue, construct or scaffold can have a printed size in the interval from 0.1 mm to 50 cm in diameter and/or length or width. The bioprinter apparatus can be of any commercially available type, such as the 3D Bioprinters' INKREDIBLE™, INKREDIBLE+™ or BIO X™ from CELLINK AB.

Typically, the method for preparing bioprinted tissues or scaffolds is performed under physiological conditions, which could vary depending on the tissue and/or the cells that are printed. More specifically, the conditions and parameters during bioprinting varies within the following intervals:

Temperature: 4° C. to 40° C.

Printing pressure: 1-200kPa.

Also, external cross-linking may be used during or after the bioprinting process such as calcium chloride solution, UV or light exposure in the wavelengths between 300 and 800 nm, preferability 365 nm, 405 nm, 425 nm, and 480 nm, or self-assembly of the biomaterial component under thermal incubation.

### Bioprinted Tissue or Organ

Other aspects provide a bioprinted tissue produced by a method described above. Bioprinted tissues produced as described herein display the tissue-specific extracellular matrix protein composition of the source tissue sample.

### Bioprinted Scaffold and Use of Bioprinted Scaffold

Another aspect provides a bioprinted human scaffold or tissue produced as described above for the use in tissue repair, for example.

For example, bioprinted scaffolds with or without cells and/or with or without known growth factors can be implanted in diseased-tissues or organs, such as tissue-patches, in order to promote tissue repair. For instance, tissue repair can be promoted by wound healing due to the capability of ECM to favor immunomodulation and therefore reducing tissue scarring in fibrotic diseases (e.g. liver fibrosis, intestinal fibrosis, fistulas, Chron's Disease, cartilage defects, etc.).

### Method for Culturing the Bioprinted Tissue and In Vitro Culture and Use of In Vitro Culture

Another aspect provides a bioprinted human scaffold or tissue produced as described above for the use in modeling human diseases, testing drugs and biomarker discovery.

Bioprinted tissue can be used to screen drugs and/or cell-based therapies. For example, bioprinted tissue with cancer cells can be exposed to chemotherapy agents, immunotherapy and/or CAR-T, NK cells.

### Method of Transplantation

Yet another aspect provides a bioprinted human scaffold or bioprinted human tissue produced as described above for use in the transplantation of a tissue or organ in an individual.

For example, a bioprinted human scaffold or bioprinted human tissue may be transplanted to an individual to replace an organ or a tissue.

### Method of Repairing or Regenerating Tissues and Organs

Another aspect of the present disclosure provides a bioprinted human scaffold or bioprinted human tissue produced as described above for use in the treatment of disease or dysfunction in a tissue or organ in an individual.

For example, a bioprinted human scaffold or bioprinted human tissue may be implanted in an individual to regenerate a complete new organ or to improve the repair of a damaged organ, or may support the organ function of the individual from outside the body.

### Method of Treating a Disease

The bioprinted scaffold or tissue may be useful in therapy, for example for the replacement or supplementation of tissue in an individual.

A method of treatment of a disease may comprise implanting a bioprinted human scaffold or bioprinted human tissue produced as described above into an individual in need thereof. The implanted bioprinted scaffold or tissue may replace or supplement the existing tissue in the individual.

The bioprinted scaffold or tissue may be used for the treatment of any one of the diseases chosen from, but not limited to: liver diseases, metabolic diseases, diabetes, heart diseases, kidney diseases, lung disease, skin defects, muscle defects, bone defects, bone and soft tissue sarcomas, lung diseases, vessels repair, intestinal diseases, fistulas, cartilage defects, retinal defects, bladder diseases, prostate diseases, tissue fibrosis (e.g. liver, kidney, intestine, lung, skin), cancer in any tissue, such as hepatocellular carcinoma, metastases in any tissue, such as the liver, colon or pancreas, colon cancer, lung cancer, liver cancer, pancreatic cancer, and cancer in any other tissue disclosed in this application, comprising using the bioprinted tissue, organ or scaffold.

### Method for Disease Modelling

The bioprinted tissue or bioprinted scaffold may be useful for disease modelling. Suitable ECM source(s) may be derived from a normal tissue sample or pathological tissue sample, as described above.

A method of disease modelling may comprise:
providing a bioprinted tissue or scaffold produced as described above, optionally bioprinting the tissue or scaffold with cells to produce a recellularised bioprinted tissue, and determining the effect of a compound, drug, biological agent, device or therapeutic intervention on the bioprinted scaffold or tissue or the cells therein.

Methods described herein may be useful in modelling tissue diseases or diseases affecting the tissue, such as tissue fibrosis, tissue cancer and metastases, tissue drug toxicity, post-transplant immune responses, and autoimmune diseases.

### Method of Diagnosis

Bioprinted scaffolds and tissues may be useful for the diagnosis of disease. Suitable bioprinted scaffolds and tissues may be derived from tissue from an individual suspected of having a disease in the tissue or organ.

A method of diagnosing disease in a human individual may comprise:
providing a bioprinted scaffold or tissues from the individual produced as described above, determining the presence and amount of one or more scaffold proteins in the sample. The presence and amount of scaffold proteins in the sample may be indicative of the presence of disease in the tissue or organ of the individual.

### Other Applications

The bioprinted scaffolds and tissues may also be useful for proteomics, biomarker discovery, and diagnostic applications. For example, the effect of a protease on the components, architecture or morphology of a bioprinted scaffold and tissue may be useful in the identification of biomarkers.

The present invention is not limited to the above-described preferred embodiments. Various alternatives, modifications and equivalents may be used. Therefore, the above embodiments should not be taken as limiting the scope of the invention, which is defined by the appending claims.

1. A perfusion device for printing a 3D construct with one or more internal channels, comprising
a housing with an internal chamber, the chamber configured to hold the 3D construct,
the chamber comprising at least one inlet or outlet configured to introduce a liquid from outside the chamber into the chamber or vice versa,
the chamber having a bottom plate configured to support the 3D construct comprising the one or more internal channels at least during printing of the 3D construct,
wherein each of the at least one inlet or outlet comprises a connector adapted to connect or align the one or more internal channels within the 3D construct to the inlet or outlet when the 3D construct is present in the chamber.

2. The perfusion device of claim 1, further comprising a cover adapted to enclose the chamber such that the chamber becomes essentially fluid-tight.

3. The perfusion device of claim 1, further comprising at least one perfusion passageway in connection with the inlet or outlet, adapted for fluid connection with the internal chamber.

4. The perfusion device of claim 1 comprising at least two inlets or outlets.

5. The perfusion device of claim 1 wherein the connector is adapted to anchor the construct in the chamber during and/or after printing of the construct.

6. The perfusion device of claim 1 wherein the connector comprises a distancing element adapted to assist a 3D printer in placing a material for the internal channels at suitable height from the bottom plate, such that the internal channels will align with the inlet or outlet.

7. The perfusion device of claim 1 wherein the cover and/or the bottom plate is at least partly transparent.

8. A kit comprising
a perfusion device according to claim 1
a biomaterial adapted to be used for bioprinting
a sacrificial bioink
optionally at least one bioprinting nozzle adapted for bioprinting the biomaterial
optionally suitable Luer connectors.

9. A method for printing a 3D construct with at least one internal channel, the method comprising
providing a chamber with at least one inlet or outlet comprising a connector adapted to connect to or align with said at least one internal channel, and said chamber further comprising a bottom plate,
optionally providing a first biomaterial layer on the bottom plate,
printing one or more channels on the bottom plate or on the first layer of biomaterial using a sacrificial ink, said one or more channels being printed such that they connect to or align with a connector,
providing a second biomaterial layer covering the one or more channels.

10. The method according to claim 9, comprising a step of providing a first biomaterial layer on the bottom plate before printing the one or more channels, and therafter printing one or more channels on the first layer of biomaterial.

11. The method according to claim 10, further comprising a step of dehydration of the biomaterial in the first biomaterial layer before printing the one or more channels and wherein the second biomaterial is adapted to rehydrate the first biomaterial layer.

12. The method according to claim 9, further comprising the step of evacuating the printed channels by suction or pressure.

13. The method according to claim 9, wherein the first and/or second biomaterial comprises a scaffold or matrix material.

14. The method according to claim 9, wherein the first and/or second biomaterial comprises living biological cells.

15. The method according to claim 9, wherein the construct is printed in a perfusion device for printing a 3D construct with one or more internal channels, wherein the perfusion device comprises:
a housing with an internal chamber, the chamber configured to hold the 3D construct,
the chamber comprising at least one inlet or outlet configured to introduce a liquid from outside the chamber into the chamber or vice versa, and
the chamber having a bottom plate configured to support the 3D construct comprising the one or more internal channels at least during printing of the 3D construct, wherein each of the at least one inlet or outlet comprises a connector adapted to connect or align the one or more internal channels within the 3D construct to the inlet or outlet when the 3D construct is present in the chamber.

16. A bioprinted construct, tissue or organ with at least one internal channel, said construct, tissue or organ being prepared by the method according to claim 9.

17. A method for treatment of liver diseases, metabolic diseases, diabetes, heart diseases, kidney diseases, lung disease, skin defects, muscle defects, bone defects, bone and soft tissue sarcomas, lung diseases, vessels repair, intestinal diseases, fistulas, cartilage defects, retinal defects, bladder diseases, prostate diseases, tissue fibrosis (e.g. liver, kidney, intestine, lung, skin), cancer in any tissue, such as hepatocellular carcinoma, metastases in any tissue, such as the liver, colon or pancreas, colon cancer, lung cancer, liver cancer, pancreatic cancer, and cancer in any other tissue, comprising perfusing a bioprinted construct, tissue or organ according to claim 16 with a fluid through at least one internal channel.

18. A method for culturing a bioprinted construct, tissue or organ according to claim 16, wherein the bioprinted tissue or organ is cultured under physiological or pathological conditions, said method comprising perfusing the construct, tissue or organ with a fluid through at least one internal channel.
